(19) United States
(12) Patent Application Publication
(10) Pub. No.: US 2017/0044414 A1
(43) Pub. Date: Feb. 16, 2017

(54) THERMAL ENERGY STORAGE AND TEMPERATURE STABILIZATION PHASE CHANGE MATERIALS COMPRISING ALKANOLAMIDES AND DIESTERS AND METHODS FOR MAKING AND USING THEM

(71) Applicant: Entropy Solutions Inc., Plymouth, MN (US)

(72) Inventors: William Rusty Sutterlin, Hoover, AL (US); Aymara M. Albury, Tuscaloosa, AL (US)

(21) Appl. No.: 15/305,652

(22) PCT Filed: Apr. 23, 2015
(86) PCT No.: PCT/US15/27368

§ 371 Date: Oct. 20, 2016

Related U.S. Application Data
(60) Provisional application No. 61/983,405, filed on Apr. 23, 2014.

(51) Int. Cl. C09K 5/06; C07C 69/28; C07C 233/18; C07C 69/36

(52) U.S. Cl. CPC C09K 5/063; C07C 69/36; C07C 69/28; C07C 233/18

(57) ABSTRACT

This invention generally relates to thermoregulation and temperature stabilization, thermal protection and insulation, and nucleating agents. In particular, in alternative embodiments, provided are organic phase change materials comprising diesters and alkanolamides. In alternative embodiments, provided are Phase Change Material (PCMs) compositions comprising diesters and alkanolamides, and methods for making and using them. In alternative embodiments, the Phase Change Material (PCMs) compositions are used for thermal energy management, including energy storage and/or temperature stabilization, in various applications such as building, automotive, packaging, garment and footwear, textiles, fabrics, synthetic fibers, foods, microcapsules and other energy storage systems.

### TECHNICAL FIELD

This invention generally relates to thermoregulation and temperature stabilization, thermal protection and insulation, and nucleating agents. In particular, in alternative embodiments, provided are organic phase change materials comprising diesters and/or alkanolamides. In alternative embodiments, provided are Phase Change Material (PCMs) compositions comprising diesters and/or alkanolamides, and methods for making and using them. In alternative embodiments, the Phase Change Material (PCMs) compositions are used for thermal energy management, including temperature stabilization, in various applications such as building, automotive, packaging, garment and footwear, textiles, fabrics, synthetic fibers, foods, pharmaceuticals, microcapsules and energy storage systems.

### BACKGROUND OF THE INVENTION

There is a general desire in all industries to increase energy efficiency. There is also a general desire to reduce the use of fossil fuel resources due to concerns over climate change and energy security. Buildings, for example, require significant amounts of energy for heating and cooling and there is a need to reduce the costs associated with thermal management. The thermal management of temperature sensitive payloads during transport can also require significant amounts of energy. In the automotive industry, there is a desire to increase efficiency and reduce the fuel usage associated with maintaining a comfortable temperature in the cabin of vehicles. In the textile industry, in particular for life and personal protection clothing, there is a desire to create fabrics and materials that maintain the temperature of the wearer in a comfortable range by managing away excess heat.

One approach of decreasing the amount of energy needed for thermal management is the use of phase change materials. A “phase change material” (PCM) is a material that stores or releases a large amount of energy during a change in state, or “phase”, e.g. crystallization (solidifying) or melting (liquefying) at a specific temperature. The amount of energy stored or released by a material during crystallization or melting, respectively, is the latent heat of that material. During such phase changes, the temperature of the material remains relatively constant. This is in contrast to the “sensible” heat, which does result in a temperature change of the material, but not a phase change.

PCMs are therefore “latent” thermal storage materials. A transfer of energy occurs when the material undergoes a phase change, e.g. from a liquid to a solid and thus helps to maintain the temperature of a system. When heat is supplied to the system in which the temperature is at the melting point of the PCM, energy will be stored by the PCM, resulting in a mediating effect on the temperature of the system. Similarly, when the temperature of the system decreases to the crystallization temperature of the PCM, the energy stored by the PCM will be released into the surrounding environment. The amount of energy stored or released by a material is a constant, and is that material's latent heat value. For example, water has a latent heat of 333 J/g. Therefore, a gram of water will release 333 J of energy to its surrounding environment during crystallization (freezing), at 0° C. without changing temperature. Similarly, a gram of frozen water will absorb 333 J of energy from its surrounding environment during melting without an increase in temperature from 0° C.

There are two primary characteristics that must be considered for a specific application of a PCM: 1) the melting/crystallization temperature of the material, and 2) the latent heat value. A high latent heat value is the most desirable characteristic of a phase change material. A high latent heat value means that the material will be able to store or release large amounts of energy during a phase change, thus reducing the quantity of supplied energy needed to heat or cool a system. A latent heat value of 160 J/g or higher is considered acceptable for a PCM material in thermal storage applications. The melting/crystallization temperature is important because every thermal storage system has a unique optimal temperature range. These two factors together inform the potential applications for a specific PCM. For example, although water has a very high latent value (333 J/g), it would not be suitable for use as a PCM in building materials, as buildings are typically maintained at temperatures around 21° C., well above the melting/crystallization temperature of water.

The majority of commercially available PCMs are salt hydrates or paraffins. Both salt hydrates and paraffins have inherent disadvantages in commercial applications. Salt hydrates, while cheap to produce, have inconsistent melting points, and have a tendency to supercool. Salt hydrates are also known to undergo significant thermal expansion and can be highly toxic and corrosive. Paraffins make suitable PCMs in that they have favorable latent heat values and consistent melting points. However, the high latent heats of paraffin-based PCMs (in excess of 230 J/g) require compositions comprising high purities of paraffins, necessitating the use of expensive processing technology. Further, paraffins are limited in their potential range of phase change temperatures, leading to the use of mixed PCM compositions with reduced latent heat values.

Other concerns with paraffins used as PCMs are social dynamics. Paraffins are made from petroleum products, which increases our reliance on crude oil. Paraffin prices have followed the unstable price of petroleum. Furthermore, petroleum derived paraffins have geopolitical consequences and contribute to the increase in carbon emissions blamed for the global warming crisis.

The widespread use of traditional PCMs has been further limited due to concerns over flammability. For example, the use of paraffin or vegetable oil-derived PCMs has been limited due to the inherent flammability of many of these materials. A need thus remains for PCMs with high latent heat and other favorable thermal and temperature storage properties that can be used in thermal energy storage and stabilization and temperature stabilization systems across a broad range of temperatures.

### SUMMARY OF THE INVENTION

In alternative embodiments, the provided are thermal energy storage and temperature and/or energy stabilization materials comprising at least one, or mixtures of, phase change materials (PCM) with favorable PCM characteristics, including high latent heats, wherein the thermal energy storage, or temperature and/or energy stabilization, material undergoes solid to liquid and liquid to solid phase change transitions. In alternative embodiments applications of the thermal energy storage, or thermal energy or temperature stabilization, materials include: building materials e.g. walls, flooring and tank devices used to moderate climates in buildings, food storage coolers or other types of coolers, devices used to keep food warm or cold, pharmaceutical storage vehicles, packaging, textiles, and the like, and, in alternative embodiments, essentially any device used to keep a substance at a relatively constant temperature between about 10° C. and 200° C., or between about 20° C. and 100° C., or between about 0° C. and 20° C., or between about −20° C. and 0° C. In alternative embodiments, provided are PCM compounds comprising diesters and/or alkanolamides.

In alternative embodiments, provided are compositions, products of manufacture, or thermal energy storage and/or temperature stabilization compounds, comprising at least one phase change material compound (PCM) selected from the group consisting of: (a) a diester, (b) an alkanolamide, and (c) a combination thereof,

wherein the thermal energy storage and temperature stabilization compound undergoes solid to liquid and liquid to solid phase change transitions,

and optionally for (c), the diester to alkanolamide (diester:alkanolamide) ratio is between about 1% to 99% diester with a corresponding 99% to 1% alkanolamide; or, in molar ratio about 10:90, 20:80; 30:70; 40:60, 50:50, 60:40, 70:30, 80:20, or 90:10, or between about 1 to 99 diester to about (the corresponding) 99 to 1 alkanolamide,

and optionally for (c), the diester and alkanolamide are present in the composition or product of manufacture in layers or laminates, for example, the diester PCM an inner or a core layer, and the alkanolamide as an outer layer, or the diester and alkanolamide are present in alternating layers.

In alternative embodiments, provided are compositions, products of manufacture, or thermal energy storage and/or temperature stabilization compounds, wherein the at least one phase change material (PCM) compound comprises a diester, and optionally 100% of the PCM in the composition, product of manufacture, or thermal energy storage and/or temperature stabilization compound is a diester, and optionally the PCM in the composition, product of manufacture, or thermal energy storage and/or temperature stabilization compound consists essentially of a diester.

In alternative embodiments, provided are compositions, products of manufacture, or thermal energy storage and/or temperature stabilization compounds, wherein the diester is the product of a reaction comprising a diacid or a diacid chloride and an alcohol. In alternative embodiments, the diacid or diacid chloride is selected from the group consisting of: ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid and diacid chlorides thereof, and a combination thereof.

In alternative embodiments, the alcohol is selected from the group consisting of: methanol, ethanol, propanol, butanol, pentanol, hexanol, heptanol, octanol, nonanol, decanol, undecanol, dodecanol, tridecanol, tetradecanol, pentadecanol, hexadecanol, heptadecanol, octadecanol, nonadecanol, icosanol, docosanol, tetracosanol, hexacosanol, octacosanol, and triacontanol, and a combination thereof.

In alternative embodiments, the diester is the product of a reaction comprising an acid or an acid chloride and a diol, and a combination thereof, and optionally the acid or acid chloride is selected from the group consisting of: formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and acid chlorides thereof, and a combination thereof.

In alternative embodiments, the diol is selected from the group consisting of: ethylene glycol, propylene glycol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol, 1,14-tertadecanediol, 1,15-pentadecanediol, 1,16-hexadecanediol, 1,17-heptadecanediol, 1, 18-octadecanediol, 1,19-nonadecanediol, 1,20-icosanediol, 1,22-docosanediol, 1,24-tetracosanediol, 1,26-hexacosanediol, 1,28-dioctacosanediol, 1,30-triacontandiol, and a combination thereof.

In alternative embodiments, the at least one phase change material (PCM) compound comprises an alkanolamide, and optionally 100% of the PCM in the composition, product of manufacture, or thermal energy storage and/or temperature stabilization compound is an alkanolamide, and optionally the PCM in the composition, product of manufacture, or thermal energy storage and/or temperature stabilization compound consists essentially of an alkanolamide.

In alternative embodiments, the alkanolamide is the product of a reaction comprising an alkanolamine and an acid or an acid chloride. In alternative embodiments, the alkanolamine is selected from the group consisting of: methanolamine, ethanolamine, propanolamine, butanolamine, pentanolamine, hexanolamine, heptanolamine, octanolamine, nonanolamine, decanolamine, undecanolamine, dodecanolamine, tridecanolamine, tetradecanolamine, pentadecanolamine, hexadecanoalamine, heptadecanolamine, octadecanolamine, nonadecacanolamine, icosanolamine, docosanolamine, tetracosanolamine, hexacosanolamine, octacosanolamine, triacontanolamine, and a combination thereof.

In alternative embodiments, the acid or acid chloride is selected from the group consisting of: formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof, and a combination thereof.

In alternative embodiments, provided are methods for making a composition, product of manufacture, or thermal energy storage and/or temperature stabilization compound, or a phase change material-comprising composition as provided herein, comprising blending or mixing the at least one organic phase change material and the organic nucleating agent or material after heating to above the melting point of both components either prior to or after they are mixed together.

In alternative embodiments, provided are nanoparticles or microcapsules comprising a phase change material-comprising composition as provided herein.

In alternative embodiments, provided are articles of manufacture, products of manufacture, a coating, a liquid, a gel, an antifreeze fluid, a fluid, an ink, an oil, a lubricant, a sealant, a paint, a textile, a cloth, a clothing, a bedding or bedding system, comprising: a nanoparticle or a microcapsule as provided herein, or, comprising a composition, product of manufacture, or thermal energy storage compound, or a phase change material-comprising composition as provided herein.

In alternative embodiments, provided are building materials, an automotive material, a packaging material, a garment, a footwear or a footwear material, a textile, a fabric, a synthetic fiber, a pharmaceutical or a food, or an energy storage system, comprising: a nanoparticle or a microcapsule as provided herein, or, comprising a composition, product of manufacture, or thermal energy storage compound, or a phase change material-comprising composition as provided herein.

In alternative embodiments, provided are a composition, a product of manufacture, or a thermal energy storage and/or temperature stabilization compound, comprising at least one phase change material compound (PCM), wherein the PCM consists essentially of a diester, or the PCM is about 100% or substantially all diester. In alternative embodiments, provided are a composition, a product of manufacture, or a thermal energy storage and/or temperature stabilization compound, comprising at least one phase change material compound (PCM), wherein the PCM consists essentially of an alkanolamide, or the PCM is about 100% or substantially all alkanolamide.

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments of the invention and are not meant to limit the scope of the invention as encompassed by the claims.

FIGS. 1 to 4, are described and discussed in detail herein; FIGS. 1 to 4 illustrate Differential Scanning calorimetry (DSC) scans of exemplary diesters that may be used for thermal energy storage and/or temperature stabilization in alternative embodiments of the invention, and show freezing temperature, melting point and latent heat:

FIG. 1 illustrates a Differential Scanning calorimetry scan of ethylene glycol distearate (a diester of ethylene glycol and stearic acid), which may be used as a Phase Change Material (PCM) in alternative embodiments of the present invention.

FIG. 2 illustrates a Differential Scanning calorimetry scan of ethylene glycol dipalmitate (a diester of ethylene glycol and palmitic acid), which may be used as a PCM in alternative embodiments of the present invention.

FIG. 3 illustrates a Differential Scanning calorimetry scan of distearyl oxalate (a diester of oxalic acid and stearyl alcohol), which may be used as a PCM in alternative embodiments of the present invention.

FIG. 4 illustrates a Differential Scanning calorimetry scan of dioctyl oxalate (a diester of oxalic acid and octanol), which may be used as PCM in alternative embodiments of the present invention.

Like reference symbols in the various drawings indicate like elements.

Reference will now be made in detail to various exemplary embodiments of the invention. The following detailed description is provided to give the reader a better understanding of certain details of aspects and embodiments of the invention, and should not be interpreted as a limitation on the scope of the invention.

### DETAILED DESCRIPTION OF THE INVENTION

In alternative embodiments, provided are organic phase change materials comprising at least one diester or alkanolamide, or only a diester or an alkanolamide, or substantially only diesters or only alkanolamides, and/or mixtures of diesters and alkanolamides. In alternative embodiments, the invention provides Phase Change Material (PCMs) compositions comprising or consisting essentially of at least one diester, alkanolamide and/or mixtures of diesters and alkanolamides, and methods for making and using them. In alternative embodiments, the Phase Change Material (PCMs) compositions are used for thermal energy management, e.g., thermal energy storage or stabilization, and/or temperature stabilization, in various applications such as building, automotive, packaging, garment and footwear, textiles, fibers, foods, pharmaceuticals, and other energy storage or temperature stabilization systems.

### Diester-Comprising Phase Change Materials

In alternative embodiments, provided are diester-comprising PCM compounds for use in thermal energy management, including thermal energy storage and stabilization, and temperature stabilization. In alternative embodiments, the diester is a product of a reaction comprising an alcohol comprising a single hydroxyl group, and a diacid (i.e. a dicarboxylic acid) comprising two carboxylic acid functional groups, or a diacid chloride with two acid chloride moieties.

The alcohol can be, for example, without limitation: methanol, ethanol, propanol, butanol, pentanol, hexanol, heptanol, octanol, nonanol, decanol, undecanol, dodecanol, tridecanol, tetradecanol, pentadecanol, hexadecanol, heptadecanol, octadecanol, nonadecanol, icosanol, docosanol, tetracosanol, hexacosanol, octacosanol, or triacontanol.

The diacid can be, for example, without limitation: ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid or the diacid chlorides thereof.

In some embodiments, provided are thermal energy storage and stabilization, and temperature stabilization, materials comprising a diester having been formed by the reaction of methanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of ethanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of propanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of butanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of pentanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of hexanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of heptanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of octanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of nonanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of decanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of undecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of dodecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of tridecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of tetradecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of pentadecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of hexadecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of heptadecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of octadecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of nonadecanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of icosanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of docosanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of tetracosanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of hexacosanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of octacosanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

In some embodiments, provided are thermal energy storage and stabilization, and temperature stabilization, materials comprising a diester having been formed by the reaction of triacontanol and a diacid or a diacid chloride that undergo solid-to-liquid and liquid-to-solid phase change transitions. The diacid may be, for example, ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid, or any diacid chloride thereof.

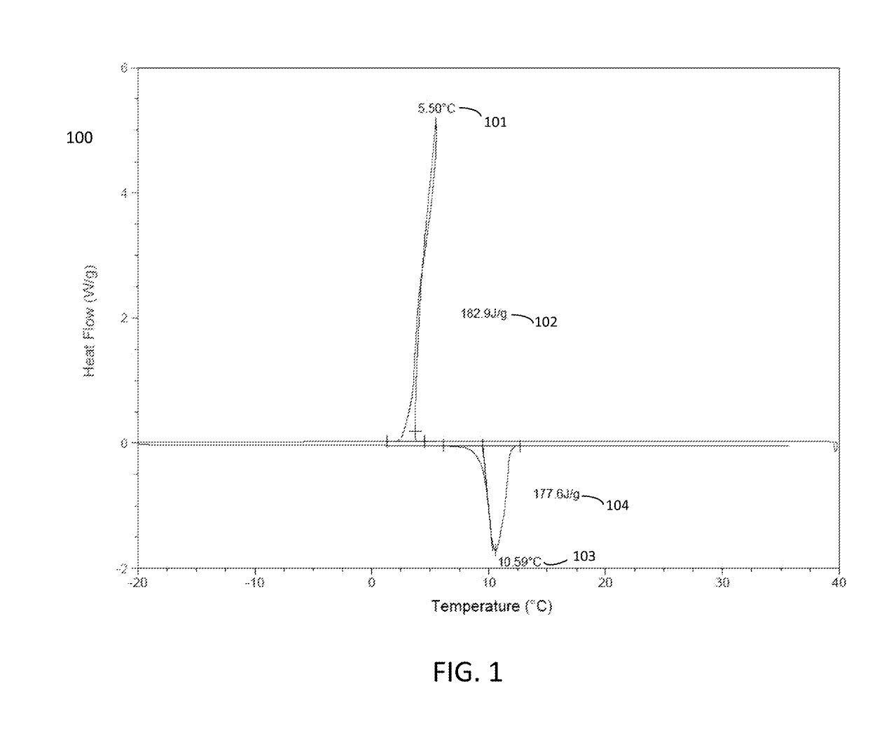

FIGS. 1 to 4 illustrate Differential Scanning calorimetry (DSC) scans of exemplary diesters that may be used for thermal energy storage and/or temperature stabilization in alternative embodiments of the invention, and show freezing temperature, melting point and latent heat. DSC scans describe: a freezing temperature of the material, as shown by a first peak with an area above the x-axis; and, a melting temperature of the material, as shown by a second peak with an area under the x-axis. Temperature differences between freezing and melting point can be due to a number of factors including supercooling and instrument functionality e.g. differences in the scan rate when measuring freezing and melting. The latent heat of the scanned material is measured by calculating the area under the curve of each of the freezing and melting peaks.

FIG. 1 shows a DSC curve 100 of ethylene glycol distearate with a measured freezing temperature of 5.50° C. 101 with a corresponding latent heat of 182.9 J/g 102, and a melting temperature of 10.59° C. 103 and a corresponding latent heat of 177.6 J/g 104.

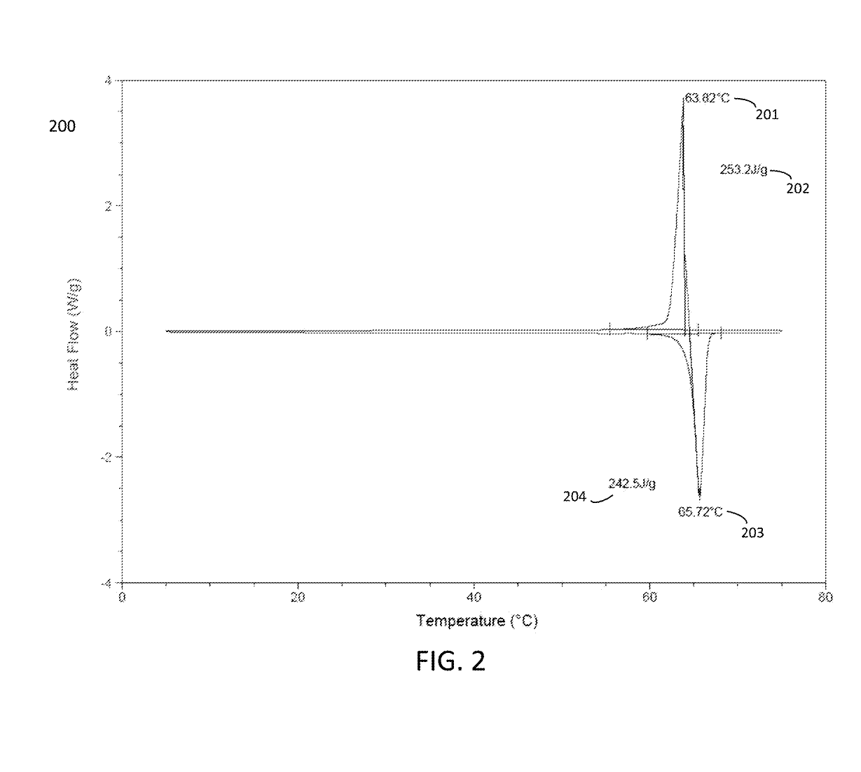

FIG. 2 shows a DSC curve 200 of ethylene glycol dipalmitate with a measured freezing temperature of 63.8° C. 201 with a corresponding latent heat of 253.2 J/g 202, and a melting temperature of 65.72° C. 203 and a corresponding latent heat of 242.5 J/g 204.

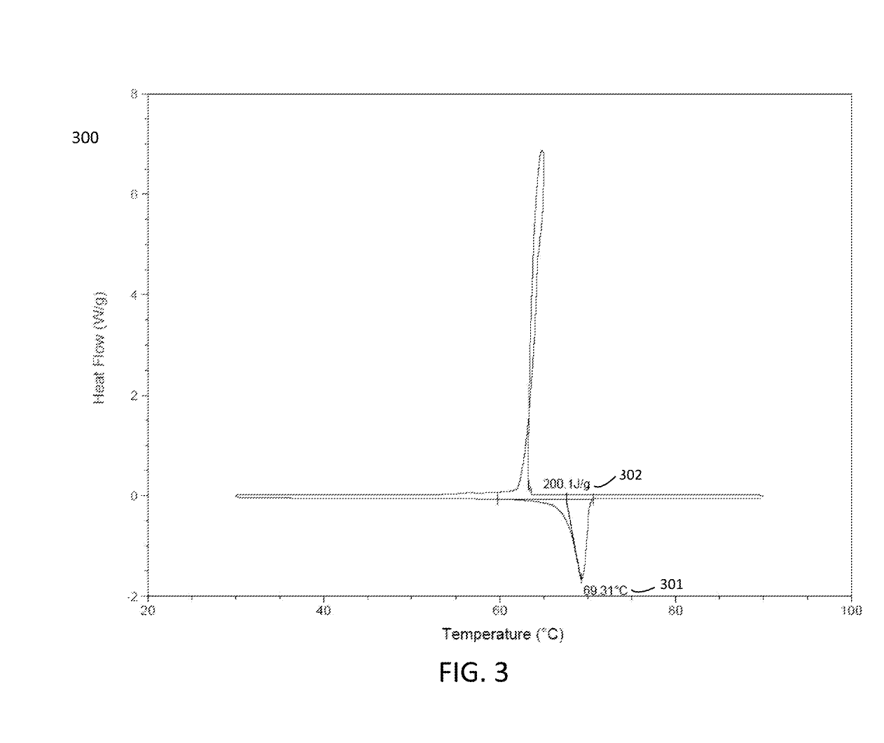

FIG. 3 shows a DSC curve 300 of distearyl oxalate with a measured melting temperature of 69.31° C. 301 and a corresponding latent heat of 200.1 J/g 302.

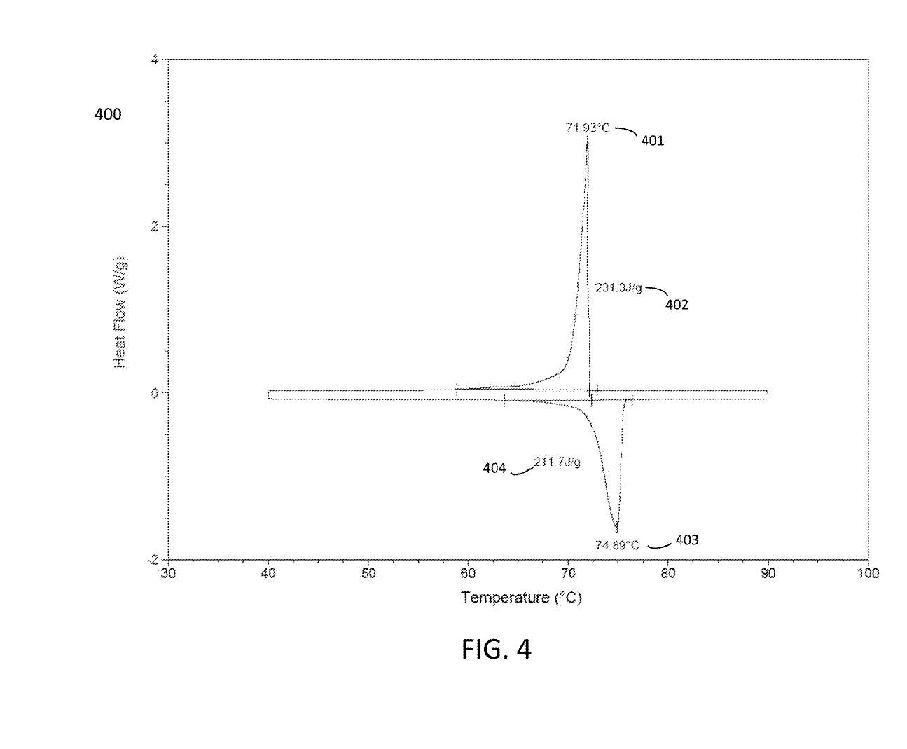

FIG. 4 shows a DSC curve 400 of dioctyl oxalate with a measured freezing temperature of 71.93° C. 401 with a corresponding latent heat of 231.3 J/g 402, and a melting temperature of 74.89° C. 403 and a corresponding latent heat of 211.7 J/g 404.

Table 1 summarizes the PCM performance of various diesters, produced by reacting a diacid or diacid chloride and an alcohol. The latent heat (joules per gram (J/g)) and melting point (° C.) of these diesters, as well as the reagents from which they were produced are provided.

TABLE 1
Diester thermal energy storage materials
and associated PCM characteristics

| | Melting | Latent | | |
|---|---|---|---|---|
| | Point | Heat | Diacid | Alcohol |
| Diester | (° C.) | (J/g) | Reagent | Reagent |
| Didecyl Oxalate | 26 | 161 | Oxalic Acid | Decyl Alcohol |
| Distearyl Oxalate | 66 | 241 | Oxalic Acid | Stearyl Alcohol |
| Distearyl Malonate | 63 | 209 | Malonic Acid | Stearyl Alcohol |
| Distearyl Succinate | 67 | 188 | Succinic Acid | Stearyl Alcohol |
| Distearyl Glutarate | 65 | 196 | Glutaric Acid | Stearyl Alcohol |
| Distearyl Adipate | 63 | 160 | Adipic Acid | Stearyl Alcohol |
| Distearyl Azelate | 63 | 182 | Azelaic Acid | Stearyl Alcohol |
| Distearyl Sebacate | 65 | 191 | Sebacic Acid | Stearyl Alcohol |

In alternative embodiments, the diester compound is a product of a reaction comprising an acid or an acid chloride, and a diol comprising two hydroxyl groups.

The acid or acid chloride can be, for example, without limitation: formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

The diol can be, for example, Ethylene glycol, propylene glycol, 1,4-butanediol, 1,5-pentanediol, 1,6-hexanediol, 1,7-heptanediol, 1,8-octanediol, 1,9-nonanediol, 1,10-decanediol, 1,11-undecanediol, 1,12-dodecanediol, 1,13-tridecanediol, 1,14-tertadecanediol, 1,15-pentadecanediol, 1,16-hexadecanediol, 1,17-heptadecanediol, 1, 18-octadecanediol, 1,19-nonadecanediol, 1,20-icosanediol, 1,22-docosanediol, 1,24-tetracosanediol, 1,26-hexacosanediol, 1,28-dioctacosanediol, 1,30-triacontandiol.

In some embodiments, provided are thermal energy storage and stabilization, and temperature stabilization, materials comprising a diester having been formed by the reaction of ethylene glycol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of propylene glycol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,4-butanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,5-pentanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,6-hexanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,7-heptanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,8-octanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,9-nonanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,10-decanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,11-undecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,12-dodecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,13-tridecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,14-tertadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,15-pentadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,16-hexadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,17-heptadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,18-octadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,19-nonadecanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,20-icosanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage or stabilization, and temperature stabilization, materials comprising a diester having been formed by the reaction of 1,22-docosanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,24-tetracosanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,26-hexacosanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,28-dioctacosanediol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising a diester having been formed by the reaction of 1,30-triacontandiol and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

Table 2 summarizes the PCM performance of various diesters, produced by reacting a diol and an acid or an acid chloride. The latent heat (joules per gram (J/g)) and melting point (° C.) of these diesters, as well as the reagents from which they were produced are provided.

TABLE 1
Diester thermal energy storage materials and associated PCM characteristics

| | Melting | Latent | Diol | Acid |
|---|---|---|---|---|
| Diester | Point (° C.) | Heat (J/g) | Reagent | Reagent |
| Ethylene Glycol Dimyristate | 62 | 237 | Ethylene Glycol | Myristic Acid |
| Ethylene Glycol Dipalmitate | 69 | 245 | Ethylene Glycol | Palmitic Acid |
| Ethylene Glycol Distearate | 76 | 247 | Ethylene Glycol | Stearic Acid |
| 1,4-butanediol dimyristate | 55 | 251 | 1,4-butanediol | Myristic Acid |
| 1,4-butanediol dipalmitate | 63 | 243 | 1,4-butanediol | Palmitic Acid |
| 1,4-butanediol distearate | 69 | 235 | 1,4-butanediol | Stearic Acid |
| 1,6-hexanediol dimyristate | 50 | 196 | 1,6-hexanediol | Myristic Acid |
| 1,6-hexanediol dipalmitate | 58 | 192 | 1,6-hexanediol | Palmitic Acid |
| 1,6-hexanediol distearate | 63 | 220 | 1,6-hexanediol | Stearic Acid |

### Alkanolamide-Comprising Phase Change Materials

In alternative embodiments, provided are alkanolamide PCM compounds for use in thermal energy management, including thermal energy storage or stabilization, and temperature stabilization. In alternative embodiments, the alkanolamide is a product of the amidation of an alkanolamine and an acid or an acid chloride.

The alkanolamine can be, for example, without limitation: Methanolamine, ethanolamine, propanolamine, butanolamine, pentanolamine, hexanolamine, heptanolamine, octanolamine, nonanolamine, decanolamine, undecanolamine, dodecanolamine, tridecanolamine, tetradecanolamine, pentadecanolamine, hexadecanoalamine, heptadecanolamine, octadecanolamine, nonadecacanolamine, icosanolamine, docosanolamine, tetracosanolamine, hexacosanolamine, octacosanolamine, or triacontanolamine.

The acid can be, for example, without limitation: Formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof.

In some embodiments, provided are thermal energy storage and stabilization, and temperature stabilization, materials comprising an alkanolamide having been formed by the reaction of methanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of ethanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of propanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of butanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of pentanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of hexanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of heptanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of octanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of nonanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of decanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of undecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of dodecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of tridecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of tetradecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of pentadecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of hexadecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of heptadecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of octadecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of nonadecanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of icosanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of docosanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of tetracosanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of hexacosonalamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of octacosanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

In some embodiments, provided are thermal energy storage and stabilization, and temperature stabilization, materials comprising an alkanolamide having been formed by the reaction of triacontanolamine and an acid or an acid chloride that undergo solid-to-liquid and liquid-to-solid phase change transitions. The acid may be, for example, formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid or any acid chloride thereof.

The following examples, and the figures, are intended to clarify the invention, and to demonstrate and further illustrate certain preferred embodiments and aspects without restricting the subject of the invention to the examples.

### EXAMPLES

### Example 1

This example describes an exemplary diester thermal energy storage, and temperature stabilization, material having favorable PCM characteristics and methods for producing the same.

Distearyl Oxalate was produced by reacting oxalyic acid with stearyl alcohol in the presence of a methanesulfonic acid catalyst. Table 3 shows the starting reagents, their molecular weights, and the mole ratio for each reagent to be used.

TABLE 3
Reagents used for the preparation of distearyl oxalate

| Compound | MW (g/mol) | Equiv. |
|---|---|---|
| Oxalic Acid | 90.04 | 1.0 |
| Stearyl Alcohol | 270.5 | 2.75 |
| Methanesulfonic Acid | 96.11 | 0.2 |

### Procedure

Oxalic acid (1.0 equivalent) and decanol (2.75 equivalent) was added to a flame dried round bottom flask equipped with stir bar. Methanesulfonic acid (0.2 equivalent) was then added dropwise to the reaction mixture. The reaction mixture was then heated to 105° C. and allowed to stir at this temperature for 15 hrs. The reaction mixture was then transferred to a beaker and allowed to cool to room temperature. The resulting solid formed by the reaction was then ground to a powder and recrystallized from 100% ethanol. Recrystallization yields a crystalline white solid.

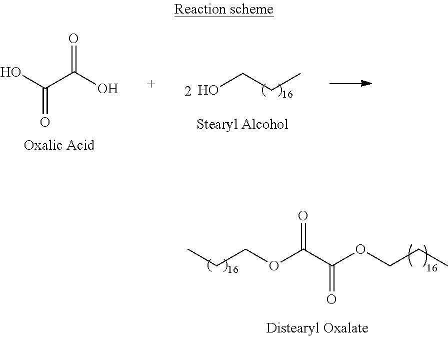

### PCM Characteristics of the Resulting Material

Melting point: 65.7° C.

Latent Heat: 249 J/g

### Exemplary Thermal Energy Storage Applications of the Distearyl Oxalate

An exemplary use of the material in the forgoing example is the incorporation of the distearyl oxalate into a heating device such as the NINA (non-instrumented isothermal nucleic acid amplification) heater, used for diagnosis of HIV-1 in point-of-care settings. The PCM is used to buffer heat from the exothermic reaction that is takes place in the heater. This provides a steady temperature of 65° C. for incubation of the test sample for reverse transcription, loop-mediated isothermal amplification.

### Example 2

This example describes an exemplary diester thermal energy storage, and temperature stabilization, material having favorable PCM characteristics and methods for producing the same.

Ethylene Glycol Distearate was produced by reacting oxalyic acid with stearyl alcohol in the presence of a methanesulfonic acid catalyst. Table 4 shows the starting reagents, their molecular weights, and the mole ratio for each reagent to be used.

TABLE 4
Reagents used for the preparation of Ethylene Glycol Distearate

| Compound | MW (g/mol) | Equiv. |
|---|---|---|
| Ethylene Glycol | 62.07 | 1.0 |
| Stearic Acid | 284.48 | 2.75 |
| Methanesulfonic Acid | 96.11 | 0.2 |

### Procedure

Ethylene glycol (1.0 equivalent) and stearic acid (2.75 equivalent) was added to a flame dried round bottom flask equipped with stir bar. Methanesulfonic acid (0.2 equivalent) was then added dropwise to the reaction mixture. The reaction mixture was then heated to 105° C. and allowed to stir at this temperature for 15 hrs. The reaction mixture was then transferred to a beaker and allowed to cool to room temperature. The resulting solid formed by the reaction was then ground to a powder and recrystallized from 100% ethanol. Recrystallization yields a crystalline white solid.

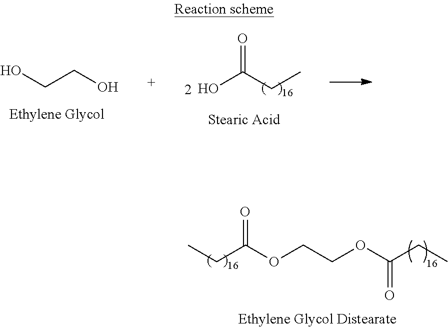

### PCM Characteristics of the Resulting Material

Melting point: 76.1° C.

Latent Heat: 247 J/g

### Exemplary Thermal Energy Storage Applications of the Ethylene Glycol Distearate

This material can be incorporated into the HVAC system of electronic cars to assist in heating the cabin. By doing so, the amount of energy that is used from the battery for cabin heating is reduced, thereby allowing for the electronic vehicle to travel further.

### Example 3

This example describes an exemplary alkanolamide thermal energy storage, and temperature stabilization, material having favorable PCM characteristics and methods for producing the same.

N-Palmitoyl ethanolamide was produced by reacting oxalyic acid with stearyl alcohol in the presence of a methanesulfonic acid catalyst. Table 4 shows the starting reagents, their molecular weights, and the mole ratio for each reagent to be used.

TABLE 4
Reagents used for the preparation of N-Palmitoyl ethanolamide

| Compound | MW (g/mol) | Equiv. |
|---|---|---|
| Palmitoyl Chloride | 274.84 | 1.0 |
| Ethanolamine | 61.08 | 10 |
| Anhydrous Tetrahydrofuran (THF) | 72.11 | 0.2 |

### Procedure

To a flame dried round bottom flask equipped with stir bar under an atmosphere of nitrogen containing a solution of palmitoyl chloride (1.0 equivalent, or eq) in anhydrous tetrahydrofuran (0.2 M) at 0° C., was added ethanolamine (10 eq) dropwise. The reaction mixture was allowed to warm to room temperature slowly and allowed to stir for 3 hrs. At this time the reaction was diluted with chloroform, washed successively with 10% HCl and 10% NaOH solutions, dried over MgSO₄ and filtered. The solvent was removed under vacuum and the solid was purified via recrystallization from ethyl acetate to afford a white solid.

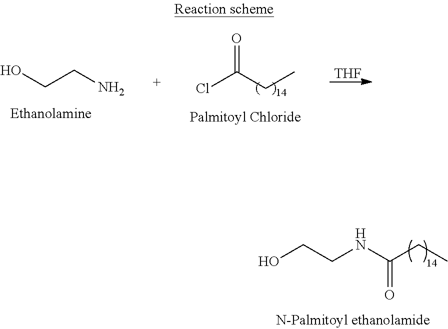

### PCM Characteristics of the Resulting Material

Melting point: 98.6° C.

Latent Heat: 140 J/g

While the forgoing written description of the invention enables one of ordinary skill to make and use what is considered presently to be the best mode thereof, those of ordinary skill will understand and appreciate the existence of variations, combinations, and equivalents of the specific embodiments, methods, and examples herein. The invention should therefore not be limited by the above described embodiments, methods and examples, but by all embodiments and methods within the scope and spirit of the invention.

A number of embodiments of the invention have been described. Nevertheless, it will be understood that various modifications may be made without departing from the spirit and scope of the invention. Accordingly, other embodiments are within the scope of the following claims.

1. A composition, a product of manufacture, or a thermal energy storage and/or temperature stabilization compound, comprising at least one phase change material compound (PCM) selected from the group consisting of:
(a) a diester,
(b) an alkanolamide, and
(c) a combination thereof,
wherein the thermal energy storage and temperature stabilization compound undergoes solid to liquid and liquid to solid phase change transitions.

2. The composition, product of manufacture, or thermal energy storage and/or temperature stabilization compound of claim 1, wherein the at least one phase change material (PCM) compound comprises a diester,
and optionally 100% of the PCM in the composition, product of manufacture, or thermal energy storage and/or temperature stabilization compound is a diester,
and optionally the PCM in the composition, product of manufacture, or thermal energy storage and/or temperature stabilization compound consists essentially of a diester.

3. The composition, product of manufacture, or thermal energy storage and/or temperature stabilization compound of claim 2, wherein the diester is the product of a reaction comprising a diacid or a diacid chloride and an alcohol,
and optionally the diester is a product of a reaction comprising an alcohol comprising a single hydroxyl group, and a diacid or a dicarboxylic acid comprising two carboxylic acid functional groups, or a diacid chloride with two acid chloride moieties,
and optionally the diester is formed by the reaction of: a methanol, an ethanol, a propanol, a butanol, a pentanol, a hexanol, a heptanol, an octanol, a nonanol, a decanol, an undecanol, a dodecanol, a tridecanol, a tetradecanol, a pentadecanol, a hexadecanol, a heptadecanol, an octadecanol, a nonadecanol, an icosanol, a docosanol, a tetracosanol, a hexacosanol, an octacosanol, a triacontanol, or a combination thereof, optionally a combination of one, two, three, four or five or more thereof; and, a diacid or a diacid chloride, optionally a diacid or diacid chloride that undergoes solid-to-liquid and/or liquid-to-solid phase change transitions,
and optionally the diester is formed by the reaction of: an ethylene glycol, a propylene glycol, a 1,4-butanediol, a 1,5-pentanediol, a 1,6-hexanediol, a 1,7-heptanediol, a 1,8-octanediol, a 1,9-nonanediol, a 1,10-decanediol, a 1,11-undecanediol, a 1,12-dodecanediol, a 1,13-tridecanediol, a 1,14-tertadecanediol, a 1,15-pentadecanediol, a 1,16-hexadecanediol, a 1,17-heptadecanediol, a 1,18-octadecanediol, a 1,19-nonadecanediol, a 1,20-icosanediol, a 1,22-docosanediol, a 1,24-tetracosanediol, a 1,26-hexacosanediol, a 1,28-dioctacosanediol, a 1,30-triacontandiol, or a combination thereof, optionally a combination of one, two, three, four our five or more thereof; and, an acid or an acid chloride, optionally a diacid or diacid chloride that undergoes that undergoes solid-to-liquid and liquid-to-solid phase change transitions.

4. The composition, product of manufacture, or thermal energy storage and/or temperature stabilization compound of claim 3, wherein the diacid or diacid chloride is selected from the group consisting of: ethanedioic acid, propanedioic acid, butanedioic acid, pentanedioic acid, hexanedioic acid, heptanedioic acid, octanedioic acid, nonanedioic acid, decanedioic acid, undecanedioic acid, dodecanedioic acid, tridecanedioic acid, tetradecanedioic acid, pentadecanedioic acid, hexadecanedioic acid, heptadecanedioic acid, octadecanedioic acid, nonadecanedioic acid, eicosanedioic acid, heneicosanedioic acid, docosanedioic acid, tricosanedioic acid, tetracosanedioic acid, pentacosanedioic acid, hexacosanedioic acid, heptacosanedioic acid, octacosanedioic acid, nonacosanedioic acid, triacontanedioic acid, henatriacontanedioic acid, dotriacontanedioic acid, tritriacontanedioic acid, tetratriacontanedioic acid, pentatriacontanedioic acid, hexatriacontanedioic acid and diacid chlorides thereof, and mixtures and combinations thereof.

5. The composition, product of manufacture, or thermal energy storage and/or temperature stabilization compound of claim 3, wherein the alcohol is selected from the group consisting of: methanol, ethanol, propanol, butanol, pentanol, hexanol, heptanol, octanol, nonanol, decanol, undecanol, dodecanol, tridecanol, tetradecanol, pentadecanol, hexadecanol, heptadecanol, octadecanol, nonadecanol, icosanol, docosanol, tetracosanol, hexacosanol, octacosanol, and triacontanol, and mixtures and combinations thereof.

6. The composition, product of manufacture, or thermal energy storage and/or temperature stabilization compound of claim 2, wherein the diester is the product of a reaction comprising an acid or an acid chloride and a diol, and optionally the diester is a product of a reaction comprising an acid or an acid chloride, and a diol comprising two hydroxyl groups.

7. The composition, product of manufacture, or thermal energy storage and temperature stabilization compound of claim 6, wherein the acid or acid chloride is selected from the group consisting of: formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and acid chlorides thereof, and mixtures and combinations thereof.

8. The composition, product of manufacture, or thermal energy storage and/or temperature stabilization compound of claim 6, wherein the diol is selected from the group consisting of: ethylene glycol, propylene glycol, 1,4-butanediol, 1,5-pentanediol, 1,6-hexanediol, 1,7-heptanediol, 1, 8-octanediol, 1,9-nonanediol, 1, 10-decanediol, 1,11-undecanediol, 1,12-dodecanediol, 1,13-tridecanediol, 1,14-tertadecanediol, 1,15-pentadecanediol, 1,16-hexadecanediol, 1,17-heptadecanediol, 1, 18-octadecanediol, 1,19-nonadecanediol, 1,20-icosanediol, 1,22-docosanediol, 1,24-tetracosanediol, 1,26-hexacosanediol, 1,28-dioctacosanediol, 1,30-triacontandiol, and mixtures and combinations thereof.

9. The composition, product of manufacture, or thermal energy storage and/or temperature stabilization compound of claim 1, wherein the at least one phase change material (PCM) compound comprises an alkanolamide,
and optionally 100% of the PCM in the composition, product of manufacture, or thermal energy storage and/or temperature stabilization compound is an alkanolamide, and optionally the PCM in the composition, product of manufacture, or thermal energy storage and/or temperature stabilization compound consists essentially of an alkanolamide.

10. The composition, product of manufacture, or thermal energy storage and/or temperature stabilization compound of claim 9, wherein the alkanolamide is the product of a reaction comprising an alkanolamine and an acid or an acid chloride, and optionally an acid or an acid chloride that undergo solid-to-liquid and/or liquid-to-solid phase change transitions.

11. The thermal energy storage compound and/or temperature stabilization of claim 10, wherein the alkanolamine is selected from the group consisting of:
methanolamine, ethanolamine, propanolamine, butanolamine, pentanolamine,
hexanolamine, heptanolamine, octanolamine, nonanolamine, decanolamine,
undecanolamine, dodecanolamine, tridecanolamine, tetradecanolamine,
pentadecanolamine, hexadecanoalamine, heptadecanolamine, octadecanolamine, nonadecacanolamine, icosanolamine, docosanolamine, tetracosanolamine,
hexacosanolamine, octacosanolamine, triacontanolamine, and mixtures and combinations thereof.

12. The composition, product of manufacture, or thermal energy storage and/or temperature stabilization compound of claim 10, wherein the acid or acid chloride is selected from the group consisting of: formic acid, acetic acid, propionic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, hexatriacontanoic acid and the acid chlorides thereof, and mixtures and combinations thereof.

13. A method for making a composition, product of manufacture, or thermal energy storage and/or temperature stabilization compound, or a phase change material-comprising composition, of claim 1, comprising blending or mixing the at least one organic phase change material and the organic nucleating agent or material after heating to above the melting point of both components either prior to or after they are mixed together.

14. A nanoparticle or a microcapsule comprising a phase change material-comprising composition of claim 1.

15. An article of manufacture, a product of manufacture, a coating, a liquid, a gel, an antifreeze fluid, a fluid, an ink, an oil, a lubricant, a sealant, a paint, a textile, a cloth, a clothing, a bedding or bedding system, comprising: a nanoparticle or a microcapsule of claim 14, or, comprising a composition, product of manufacture, or thermal energy storage compound, or a phase change material-comprising composition of claim 1.

16. A building material, an automotive material, a packaging material, a garment, a footwear or a footwear material, a textile, a fabric, a synthetic fiber, a pharmaceutical or a food, or an energy storage system, comprising: a nanoparticle or a microcapsule of claim 14, or, comprising a composition, product of manufacture, or thermal energy storage compound, or a phase change material-comprising composition of claim 1.

17. A composition, a product of manufacture, or a thermal energy storage and/or temperature stabilization compound, comprising at least one phase change material compound (PCM), wherein the PCM consists essentially of a diester, or the PCM is about 100% or substantially all diester.

18. A composition, a product of manufacture, or a thermal energy storage and/or temperature stabilization compound, comprising at least one phase change material compound (PCM), wherein the PCM consists essentially of an alkanolamide, or the PCM is about 100% or substantially all alkanolamide.

19. The composition, product of manufacture, or thermal energy storage and/or temperature stabilization compound of claim 1, wherein for step (c) the diester to alkanolamide (diester:alkanolamide) ratio is between about 1% to 99% diester with a corresponding 99% to 1% alkanolamide; or, in molar ratio about 10:90, 20:80; 30:70; 40:60, 50:50, 60:40, 70:30, 80:20, or 90:10, or between about 1 to 99 diester to about (the corresponding) 99 to 1 alkanolamide,

20. The composition, product of manufacture, or thermal energy storage and/or temperature stabilization compound of claim 1, wherein for step (c) the diester and alkanolamide are present in the composition or product of manufacture in layers or laminates, for example, the diester PCM an inner or a core layer, and the alkanolamide as an outer layer, or the diester and alkanolamide are present in alternating layers.
